(19) United States
(12) Patent Application Publication
(10) Pub. No.: US 2024/0246880 A1
(43) Pub. Date: Jul. 25, 2024

(54) ORGANIC FERTILIZER GENERATION

(71) Applicant: Biological Organic Solutions, LLC, Bountiful, UT (US)

(72) Inventors: Cody James Vavra, Parker, CO (US); Paul Thomson Baker, Holladay, UT (US); Robert Walter Frachetti, Denver, CO (US); Larry Scott Rogers, Bountiful, UT (US)

(21) Appl. No.: 18/158,297

(22) Filed: Jan. 23, 2023

(51) Int. Cl. C05F 17/20; C05F 11/08; C05F 17/80

(52) U.S. Cl. CPC C05F 17/20; C05F 11/08; C05F 17/80

(57) ABSTRACT

A method of generating organic fertilizer material may include generating biologically produced sulfuric acid by passing an aqueous solution including sulfur over a material that may include sulfur oxidizing microorganisms. The operations may further include, recirculating the aqueous solution over the material until an amount of biologically produced sulfuric acid in the aqueous solution may reach a predefined threshold. The operations may further include extracting the biologically produced sulfuric acid from the aqueous solution. Further, the operations may include generating the organic fertilizer material using the extracted biologically produced sulfuric acid and an organic feedstock.

### FIELD

The embodiments discussed in the present disclosure are related to organic fertilizer generation.

### BACKGROUND

Unless otherwise indicated in the present disclosure, the materials described in the present disclosure are not prior art to the claims in the present application and are not admitted to be prior art by inclusion in this section.

Organic forward osmosis systems and processes provide for the combined and simultaneous treatment of filtrate wastewater from anaerobic digestion, reverse osmosis membrane water treatment waste brines, and the production of high-grade organic fertilizer.

Additionally, Sulfuric acid (H₂SO₄) is a widely used mineral acid which is produced and consumed in large quantities. Sulfuric acid is widely applied in agriculture in soil acidification. A high pH limits nutrient supply and plant growth. Sulfuric acid can be supplied to the soil to lower the soil pH and consequently improve crop performance.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One or more embodiments of the present disclosure may include a method. The method may include generating biologically produced sulfuric acid by passing an aqueous solution over a material including sulfur oxidizing microorganisms. In some embodiments, the aqueous solution may include sulfur. The method may additionally include recirculating the aqueous solution over the material until an amount of biologically produced sulfuric acid in the aqueous solution may reach a predefined threshold. Additionally, the method may include extracting the biologically produced sulfuric acid from the aqueous solution. The method may additionally include generating a fertilizer material using the extracted biologically produced sulfuric acid and an organic feedstock.

Another embodiment of the present disclosure may include a method. The method may include running a filtrate stream on a first side of a forward osmosis membrane. In some embodiments, the method may include running a draw solution on a second side of the forward osmosis membrane. In some embodiments, the draw solution may include biologically produced sulfuric acid that may be generated using sulfur oxidizing microorganisms. The method may additionally include separating, through the forward osmosis membrane, a first stream that may include a forward osmosis concentrate, where the forward osmosis concentrate may include one or more of phosphorus, nitrogen, potassium, or other trace minerals. In some embodiments, the method may include a second stream that may include one or more of water and ammonium salt. The method may additionally include generating a fertilizer product using reverse osmosis. In some embodiments, the reverse osmosis may include running the second stream through a reverse osmosis membrane that may produce a third stream and a fourth stream. In some embodiments, the third stream may include water and the fourth stream may include the fertilizer product.

Additionally, one or more embodiments of the present disclosure may include a method. The method may include providing, producing, or otherwise obtaining an aqueous solution that may include sulfur particles. In some embodiments, the sulfur particles may be pretreated such that at least a portion of the sulfur particles are no greater than fifteen (15) microns. Additionally, the method may include passing the aqueous solution over a filter including a biofilm of sulfur oxidizing microorganisms. In some embodiments, the sulfur oxidizing microorganisms may generate biologically produced sulfuric acid from the sulfur in the aqueous solution. The method may further include recirculating the aqueous solution over the filter including the biofilm until an amount of biologically produced sulfuric acid in the aqueous solution may reach a predefined threshold. The method may further include extracting the biologically produced sulfuric acid from the aqueous solution. Further, the method may include running a filtrate stream on a first side of a forward osmosis membrane. In some embodiments, the filtrate stream may include an organic feedstock. In some embodiments, the filtrate stream may include one or more streams and/or solutions that may be generated using at least the organic feedstock or any material and/or compound that may be derived using the organic feedstock. Additionally or alternatively, a draw solution may be run on a second side of the forward osmosis membrane. In some embodiments, the draw solution may include the extracted biologically produced sulfuric acid. The method may further include separating, through the forward osmosis membrane, a first stream that may include a forward osmosis concentrate that may include one or more of phosphorus, nitrogen, and potassium. In some embodiments, a second stream may include at least water and ammonium salt. The method may additionally include running the second stream through one or more reverse osmosis membranes that may produce water separated out from a fertilizer product. Additionally, the method may include generating an enhanced fertilizer product by combining at least a portion of the forward osmosis concentrate and at least a portion of the fertilizer product.

The object and advantages of the embodiments will be realized and achieved at least by the elements, features, and combinations particularly pointed out in the claims. It is to be understood that both the foregoing general description and the following detailed description are exemplary and explanatory and are not restrictive of the invention, as claimed.

### BRIEF DESCRIPTION OF THE DRAWINGS

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates an example environment of generating an organic fertilizer product, in accordance with some embodiments of the present disclosure;

FIG. 2 illustrates an example environment related to generating an organic draw solution including organically generated biologically produced sulfuric acid, in accordance with some embodiments of the present disclosure;

FIG. 3 illustrates an example environment of generating a filtrate using one or more organic feedstocks, in accordance with some embodiments of the present disclosure;

FIG. 4 illustrates an example environment of generating an organic fertilizer product and/or an enhanced organic fertilizer product using a filtrate and a draw solution, in accordance with some embodiments of the present disclosure; and

FIG. 5A illustrates a flowchart of an example method of generating fertilizer material, in accordance with at least one embodiment described in the present disclosure.

FIG. 5B illustrates a flowchart of an example method of generating an enhanced fertilizer product, in accordance with at least one embodiment described in the present disclosure.

The elements of the figures are arranged in accordance with at least one of the embodiments described herein, and which arrangement may be modified in accordance with the disclosure provided herein by one of ordinary skill in the art.

### DETAILED DESCRIPTION

Systems, processes, and methods relating to generating one or more organic fertilizers and/or organic fertilizer material are disclosed in the present disclosure. Fertilizers are widely used to cultivate crops, increase yields, promote soil health, increase usability of farming land, etc. Additionally, in recent years the demand for sustainable and organic fertilizers has increased. In many instances, certifiably organic fertilizers, products, and processes are demanded from clients, consumers, governments, and others. However, as demand for organic fertilizer increases, creating sustainable, certifiably organic fertilizers to meet the demand becomes increasingly difficult. For starters, not only must the final product be organic, but the compounds used to create the final product must also meet various organic standards.

As used in the present disclosure, “organic,” may refer to meeting requirements set forth by U.S. regulatory bodies (e.g., the United States Department of Agriculture “USDA”, the United States Environmental Protection Agency “EPA,” etc.), or other foreign regulatory bodies. The term organic may be used to identify one or more instances wherein one or more products and/or processes described in the present disclosure may be certified by the regulatory body as organic. For example, products and/or processes may meet requirements set forth in 37 C.F.R. §§ 205.1-205.7. Further, the products and/or processes described herein may be certifiably organic according to other regulatory bodies across the globe (e.g., regulatory agencies in the European Union, Mexico, Canada, etc.).

One or more embodiments disclosed herein may relate to the production of one or more organic fertilizer materials that may be produced using one or more organic feedstocks. In some instances, organic feedstocks from animal waste, green waste, food waste, and other organic wastes may include high concentrations of ammoniacal nitrogen and other plant nutrients, making organic feedstock a desirable medium for generating fertilizer. Embodiments of the present disclosure may include processes that may recover a major portion of the nitrogen and other important plant nutrients in a concentrated form as an organic liquid or solid fertilizer.

In some instances, to produce one or more fertilizers using the organic feedstock, the organic feedstock may undergo mechanical coarse solids separation to remove larger solids. In some instances, the larger solids separated from the organic feedstock may be composted or used as bedding material. In some instances, separating the coarse solids may be accomplished using one or more machines, devices, and/or processes that may be configured to separate solids. By way of example and not limitation, coarse solids may be separated from the organic feedstock using one or more of a screw press, a centrifuge, a screen, etc. In some instances, after the coarse solids are removed from the organic feedstock, fine solids (e.g., solids smaller than the coarse solids removed during the coarse solids separation step) may be removed using one or more machines, devices, and/or processes that may be configured to separate solids. By way of example and not limitation, fine solids may be separated from the organic feedstock using a membrane, a filter, a screen, dissolved air floatation (DAF), etc. In some instances, the fine solids that may have been removed from the organic feedstock may be composted, sent to further treatment, and/or recycled back to an anaerobic digester. In some instances, the organic feedstock may be pretreated using a biological and/or thermal hydrolysis process that may further degrade the organic solids for increasing biogas production and higher ammoniacal nitrogen recoveries. In some instances, one or more organic polymers may be added to the organic feedstock to assist in solids recovery in both the coarse solids separation step and the fine solids separation step.

In some instances, an aqueous solution may be produced as a filtrate, the filtrate including remaining solids and compounds in the organic feedstock after having gone through the coarse solids separation step and/or the fine solids separation step. In some instances, the filtrate may then be used with a bacterial solution containing oxidized sulfur to recover and concentrate nitrogen and other plant nutrients into a fertilizer product.

Additionally or alternatively, in some instances, a filtrate may remain as a result of the coarse solids separation step and the fine solids separation step. For example, water may be added to aid in separating the coarse and/or the fine solids from the organic feedstock, resulting in a filtrate that may include an aqueous solution. In these and other instances, the filtrate may then be sent to a forward osmosis system that may include a forward osmosis membrane. In some instances, the forward osmosis membrane may use a bacterial solution produced by sulfur oxidizing bacteria as a draw solution to concentrate the remaining suspended and dissolved solids, while selectively filtering water and dissolved compounds from the filtrate through the membrane into the draw solution. In some instances, the bacterial solution produced in an apparatus containing oxidized sulfur may be added directly to adjust the pH of an organic source or streams derived from organic sources to aid in the recovery and or concentration of nitrogen and other plant nutrients.

In some instances, the sulfur oxidizing bacteria may be grown and/or produced in an apparatus including water and a sulfur containing feedstock, and where the sulfur containing feedstock may be aerated. In some instances, other feedstocks may be used to generate organic, biologically produced sulfuric acid; for example, elemental sulfur, sulfur bentonite, micronized sulfur, and/or sulfur in biogas derived from organic feedstocks to cultivate sulfur oxidizing bacteria. In some instances, a seed culture of bacteria may be added to the sulfur feedstock and recirculated through the apparatus, while being aerated. In these and other instances, the sulfur bacteria may oxidize the sulfur and increase in population. In some instances, the biologically produced sulfuric acid that may be grown and/or produced using sulfur oxidizing bacteria may be certified as organic.

In some instances, the sulfur feedstock used to generate organic biologically produced sulfuric acid may be pretreated to reduce a particle size of at least a portion of sulfur particles. In some instances, pretreating the sulfur may include dry and/or wet milling, cavitation, high shear mixing, etc. In some instances, pretreating the sulfur may increase a surface area and/or a solubility of sulfur and/or sulfur compounds that may correspondingly increase bacterial oxidation reaction kinetics in the apparatus.

In some instances, the draw solution including a bacterial solution produced by sulfur oxidizing bacteria may become diluted during the forward osmosis process whereby water and other compounds may be pulled through the forward osmosis membrane. In some instances, the bacterial solution or draw solution may include or exclude a portion of the sulfur oxidizing bacteria (e.g., for use in the draw solution). It should be recognized that, in some instances, the bacterial solution produced by sulfur oxidizing bacteria may omit the actual bacteria and may be the product of such bacteria but may also include the bacteria in other instances. In some instances, the diluted draw solution from the forward osmosis membrane may be concentrated and/or re-concentrated using a reverse osmosis membrane separating water from the bacterial solution. In some instances, the reverse osmosis membrane process and bacterial solution that may include plant nutrients (e.g., ammoniacal nitrogen) may be used as a fertilizer product and/or recycled back to the forward osmosis membrane. In some instances, new bacterial draw solution may be added to the recycled draw solution for increased nitrogen recovery. In some instances, the draw solution, or a portion of the draw solution, may be filtered to remove at least a portion of any remaining particulate solids.

In some instances, a part of the remaining forward osmosis concentrate leaving the forward osmosis membrane may or may not be blended into the wasted bacterial draw solution containing concentrated ammoniacal nitrogen that may add micronutrients to the fertilizer product. The forward osmosis concentrate may be composted, used for irrigation, recycled back to the digester, and/or used as a fertilizer containing nitrogen, phosphorus, potassium, and other trace minerals. The water leaving the reverse osmosis as permeate can be recycled onsite.

In some instances, the forward osmosis membrane concentrate may be aerated and may correspondingly precipitate phosphorus compounds that may be collected and added into to the nitrogen fertilizer product. Additionally or alternatively, the collected precipitate phosphorus compounds may be used as an independent organic fertilizer product.

Other techniques for producing fertilizer material from organic feedstocks using forward osmosis and reverse osmosis processes may provide some fertilizer material, however, those techniques may not be certifiably organic which may be due, in part, to the use of one or more acids (e.g., sulfuric acid) as a part of a process to extract nitrogen, for example. By contrast, the fertilizer material here may use organic, biologically produced sulfuric acid using sulfur oxidizing microorganisms. The use of organically generated acids may provide the benefits of producing fertilizer products using certifiably organic products and processes.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

These and other embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise.

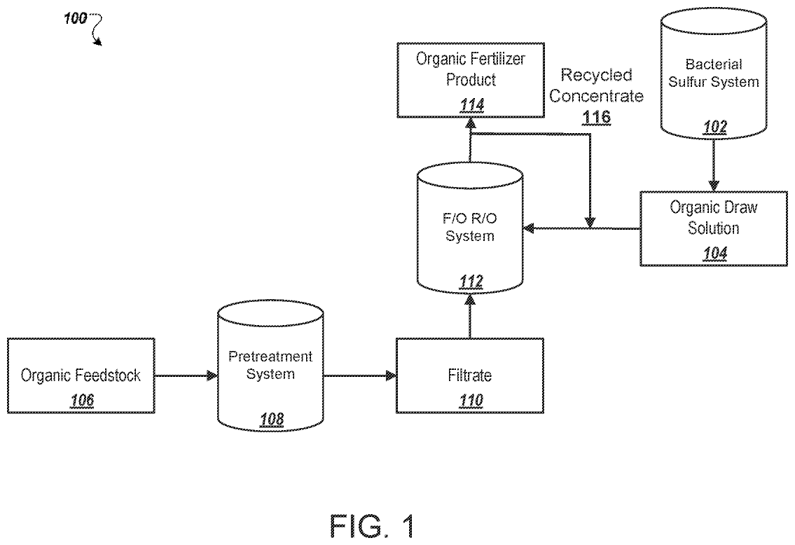

FIG. 1 illustrates an example environment 100 of generating an organic fertilizer product 114, in accordance with some embodiments of the present disclosure. In the illustrated example, the environment 100 may include a bacterial sulfur system 102, a forward osmosis and reverse osmosis (F/O R/O) system 112, and a pretreatment system 108. In some embodiments, the bacterial sulfur system 102 may generate an organic draw solution 104. Additionally, in some embodiments, the pretreatment system 108 may perform one or more operations on an organic feedstock 106 that may generate and/or produce the filtrate 110. In some embodiments, the F/O R/O system 112 may perform one or more operations to generate an organic fertilizer product 114 based at least on the filtrate 110 and the organic draw solution 104. In some embodiments, the F/O R/O system 112 may additionally recycle at least a portion of the organic fertilizer product 114 as recycled concentrate 116 to re-concentrate the organic draw solution 104.

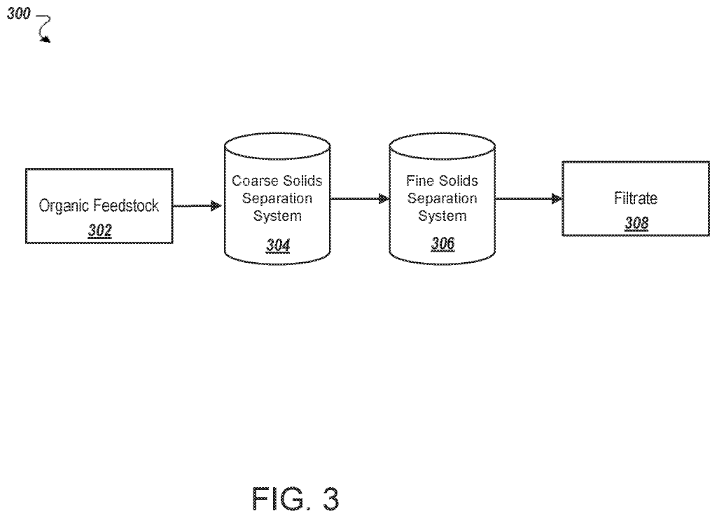

The organic feedstock 106 may include one or more organic wastes. In some embodiments, the organic feedstock 106 may include animal manure (e.g., cow manure, chicken manure, turkey manure, swine manure, and/or other animal manure), animal waste (e.g., fish emulsion, bones, blood, tissue, and other animal waste), green waste (e.g., kelp, long beans, algae, duckweed, grasses, soy derivatives, and other green waste), and/or any other organic material that may be derived from animals and/or plants. In these and other embodiments, the organic feedstock 106 may be the same and/or include an organic feedstock 302 as described and illustrated with respect to FIG. 3 in the present disclosure. In some embodiments, the organic feedstock 106 may be used in one or more systems and/or processes to generate the organic fertilizer product 114; for example, in some embodiments, the organic feedstock 106 may be used by the pretreatment system 108 to produce the filtrate 110.

The pretreatment system 108 may be configured to perform one or more operations on the organic feedstock 106 that may generate the filtrate 110 (e.g., a sludge, slurry, aqueous solution, other solutions that at least include a portion of the organic feedstock 106, and/or other solutions that may be derived using the organic feedstock 106). In some embodiments, the pretreatment system 108 may be configured to break down the organic feedstock 106 to prepare the organic feedstock for use in the F/O R/O system 112. For example, the pretreatment system 108 may separate out coarse solids and/or fine solids from the organic feedstock 106. In these and other embodiments, the pretreatment system 108 may be the same as and/or include a coarse solids separation system 304 and/or a fine solids separation system 306 as further described and illustrated with respect to FIG. 3 in the present disclosure.

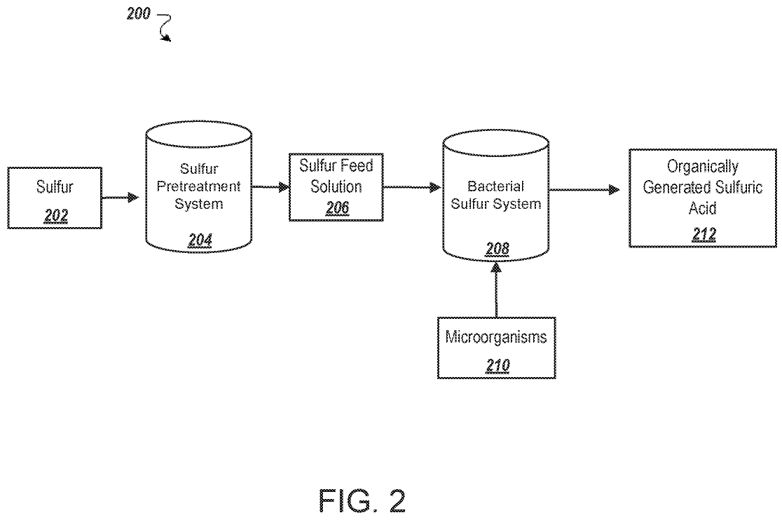

The bacterial sulfur system 102 may be configured to produce an organic draw solution (e.g., organic draw solution 104) that may contain biologically produced sulfuric acid grown, produced, and/or generated using sulfur oxidizing microorganisms. In some embodiments, a bacterial solution including organic, biologically produced sulfuric acid may be generated by the bacterial sulfur system 102. In some embodiments, the bacterial sulfur system 102 may be the same as and/or include the bacterial sulfur system 208 described and illustrated in FIG. 2 in the present disclosure.

The organic draw solution 104 may include biologically produced sulfuric acid generated using sulfur oxidizing microorganisms — e.g., sulfur oxidizing microorganisms described with respect to a bacterial sulfur system 208 and/or an organic, biologically produced sulfuric acid 212 described in the present disclosure with respect to FIG. 2. Additionally or alternatively, the organic draw solution 104 may be further configured for use in the F/O R/O system 112; for example, to create osmotic potential across a forward osmosis membrane. In these and other embodiments, the organic draw solution 104 and/or processes used to generate the organic draw solution 104 may be certified as organic.

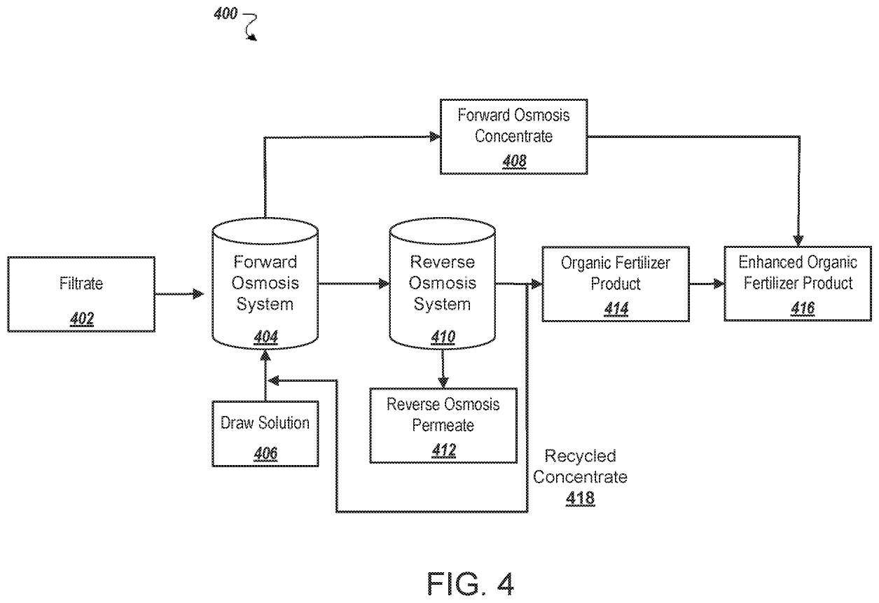

The F/O R/O system 112 may be configured to generate the organic fertilizer product 114 using the organic draw solution 104 and the filtrate 110 to generate an organic fertilizer product 114. In some embodiments, the F/O R/O system 112 may only include a forward osmosis system such as, for example, the forward osmosis system 404 described with respect to FIG. 4 in the present disclosure. In some embodiments, the F/O R/O system 112 may only include a reverse osmosis system such as, for example, the reverse osmosis system 410 described with respect to FIG. 4 in the present disclosure. In some embodiments, the F/O R/O system 112 may include both a forward osmosis system and a reverse osmosis system (e.g., the forward osmosis system 404 and the reverse osmosis system 410).

For example, the F/O/R/O system 112 may be configured to pass the filtrate 110 on a first side of a forward osmosis membrane and pass the organic draw solution 104 on a second side of the forward osmosis membrane. Continuing the example, water and other compounds may pass through the forward osmosis membrane from the filtrate 110 to the organic draw solution 104 leaving one or more of potassium, phosphorus, nitrogen, and other solids that may be used as fertilizer. Further continuing the example, the organic draw solution 104 may additionally pass through a reverse osmosis membrane in the F/O R/O system 112 separating the water from the organic draw solution 104. Continuing the example, what remains may be the organic fertilizer product 114 that may include one or more of ammonia, ammonium salt, biologically produced sulfuric acid, and/or other organic compounds used in fertilizers. Additionally or alternatively, the organic fertilizer product 114 may include the potassium, phosphorus, nitrogen, and other trace minerals, etc. from the filtrate 110 remaining after the forward osmosis process. In these and other embodiments, the organic fertilizer product 114 and/or processes, devices, resulting products, etc. may be certified as organic.

In some embodiments, the organic fertilizer product 114 may be recycled, in whole or in part, as indicated by recycled concentrate 116. In some embodiments, the recycled organic fertilizer product (e.g., recycled concentrate 116) may reconcentrate the draw solution 104 for use in the F/O R/O system 112. In these and other embodiments, the recycled concentrate 116 may increase the draw strength of a diluted draw solution as described and illustrated further with respect to the recycled concentrate 418, the reverse osmosis system 410, and/or the forward osmosis system 404 in FIG. 4 of the present disclosure.

FIG. 2 illustrates an example environment 200 related to producing an organic, biologically produced sulfuric acid 212, in accordance with some embodiments of the present disclosure. In the illustrated example, the environment 200 may include a sulfur pretreatment system 204 and a bacterial sulfur system 208. In some embodiments, the sulfur pretreatment system 204 may perform one or more operations using sulfur 202 to generate one or more outputs for use by the bacterial sulfur system 208 including, for example, a sulfur feed solution 206. Additionally, the bacterial sulfur system 208 may perform one or more operations on a sulfur feed solution 206 and microorganisms 210 from which the organic, biologically produced sulfuric acid 212 may be produced.

In some embodiments, the sulfur pretreatment system 204 may include one or more systems and/or components that may be configured to perform operations to pretreat the sulfur 202 for use in the bacterial sulfur system 208. In some embodiments, “pretreat” may refer to performing one or more operations that may reduce a particle size of at least a portion of the sulfur 202. For example, the sulfur pretreatment system 204 may reduce the particle size of at least a portion of the sulfur 202 to a lateral dimension of fifteen (15) microns or smaller. In these and other embodiments, the sulfur particle size may be tailored to specific implementations of producing the organic, biologically produced sulfuric acid 212.

In some embodiments, the particle size of at least a portion of the sulfur 202 may be reduced using one or more mechanical milling processes. For example, the sulfur pretreatment system 204 may include a high-shear mixer that may include a rotor or impeller used in a stator. Additionally or alternatively, the high shear mixer may include an array of rotors and stators used in a tank to mix a solution; in this case, the solution including sulfur 202. Further continuing the example, the high shear mixer may mix the solution to decrease the particle size of the sulfur 202.

In some embodiments, the pretreatment system may pretreat at least a portion of the sulfur 202 using one or more processes involving fluid dynamics—e.g., a high shear mixer and/or a cavitator. For example, a solution including the sulfur 202 may be fed into the cavitator, the cavitator designed to produce a controlled cavitation, where rapidly creating and collapsing bubbles may break up particles in the sulfur 202. Continuing the example, at least a portion of the resulting sulfur particles may be smaller than those entering the cavitator. Further, in some embodiments, the sulfur pretreatment system 204 may perform one or more operations on the sulfur 202 that may include a combination of the aforementioned processes—e.g., mechanical milling, high shear mixing, cavitation, etc.

Additionally or alternatively, the pretreatment system 204 may be configured to generate a solution including the sulfur 202 that may have been pretreated and/or refined using the pretreatment system 204. For example, in the context of pretreating the sulfur 202 using wet milling, water may have already been added to the sulfur 202 creating a solution. In some embodiments, when the sulfur 202 is treated without an introduction of water or other liquids, the pretreatment system 204 may add water and/or other liquids to the sulfur 202 to generate the sulfur feed solution 206. In these and other embodiments, the sulfur feed solution 206 may include one or more of elemental sulfur (S2), sulfur bentonite, hydrogen sulfide, and sulfur dioxide. In these and other embodiments, the sulfur 202 may be dissolved in water and/or other liquids prior to introducing the solution into the bacterial sulfur system 208.

In some embodiments, a particle size of at least a portion of the sulfur 202 may not be pretreated prior to introduction into the bacterial sulfur system 208. For example, some embodiments may include sulfur particles that may be larger than fifteen (15) microns. In some embodiments, the sulfur 202 may be directly added to the bacterial sulfur system 208 without having been pretreated and without preparing the sulfur feed solution 206.

The bacterial sulfur system 208 may be configured to perform one or more operations using the sulfur (e.g., the sulfur 202 and/or the sulfur feed solution 206) using the microorganisms 210 to produce the organic, biologically produced sulfuric acid 212. In these and other embodiments, the organic, biologically produced sulfuric acid 212 may be generated and/or produced using the microorganisms 210 that may be capable of oxidizing sulfur to biologically produced sulfuric acid.

For example, the bacterial sulfur system 208 may be generally configured to pass an aqueous solution including sulfur (e.g., the sulfur feed solution 206) over an aerated pile of material where the material may include microorganisms that may be capable of converting sulfur to biologically produced sulfuric acid (e.g., the microorganisms 210). Further continuing the example, the bacterial sulfur system 208 may be capable of recycling and/or returning part of the aqueous solution back over the aerated pile of material to reintroduce and/or re-expose the sulfur 202 in the sulfur feed solution 206 to the microorganisms 210. Continuing the example, the sulfur feed solution 206 may continually pass through the aerated pile of material, thereby increasing an amount of an acid product (e.g., biologically produced sulfuric acid) in the sulfur feed solution 206. Further, in some instances, the sulfur feed solution 206 may be continually passed over the aerated pile of material including the microorganisms 210 until an amount of biologically produced sulfuric acid in the sulfur feed solution 206 meets a predetermined threshold.

In some embodiments, the microorganisms 210 that may be capable of oxidizing sulfur to biologically produced sulfuric acid may include bacteria, such as bacteria belonging to the Thiobacillus genus, such as Thiobacillus thiooxidans. In some embodiments, the biologically produced sulfuric acid generated using bacteria capable of oxidizing sulfur and/or the process of generating the organic, biologically produced sulfuric acid may be certifiably organic.

Specific implementations of the bacterial sulfur system 208 generating an organic draw solution including the organic, biologically produced sulfuric acid 212 may be described in more detail below.

In some embodiments, for example, the bacterial sulfur system 208 may include: trickling a sulfur fed suspension over a filter material that may be covered with a biofilm of microorganisms capable of oxidizing sulfur to biologically produced sulfuric acid (e.g., Thiobacillus thiooxidans), collecting the trickled sulfur fed suspension in a reservoir, recirculating the collected sulfur fed suspension back to the trickling step, and eventually collecting an organic draw solution that may include biologically produced sulfuric acid.

In a starting phase of the biologically produced sulfuric acid production process, the filter material may be grafted with microorganisms (e.g., the microorganisms 210). In some embodiments, the microorganisms may be supplied with nutrients that may allow a biofilm to form on the filter material. In some embodiments, forming the biofilm on the filter material prior to beginning the biologically produced sulfuric acid production process may enable a quicker start of the process of producing biologically produced sulfuric acid from the sulfur fed suspension than simply feeding a suspension including both microorganisms and sulfur through the filter material and developing a biofilm on the filter material over time. While forming the biofilm on the filter material prior to feeding the sulfur fed solution through the filter may increase the speed through which biologically produced sulfuric acid may be produced, both embodiments are contemplated herein.

In some embodiments, the sulfur fed suspension that may be trickled over the filter material may include the sulfur fed solution 206 including the sulfur 202 that may have been used in the pretreatment system 204. In some embodiments, the sulfur 202 may not have been pretreated and the sulfur fed suspension may only include the sulfur 202 and water. In some embodiments, the sulfur fed suspension may include the sulfur feed solution 206 that may have a particle size of about fifteen (15) microns or less. In some embodiments, the use of pretreated sulfur in the bacterial sulfur system 208 may increase bacterial kinetics for the sulfur oxidation reaction. For example, using pretreated sulfur with a smaller particle size than the sulfur 202 may increase an ability of the bacteria or biofilm to interact with the pretreated sulfur such that an increased amount of biologically produced sulfuric acid may be produced. Continuing the example, using the pretreated sulfur may additionally increase a yield of biologically produced sulfuric acid per unit time as compared to using the sulfur 202 that may not have been pretreated using the pretreatment system 204. Additionally or alternatively, the use of pretreated sulfur with a smaller particle size than the sulfur 202 may additionally decrease an amount of buildup on the filter and/or other materials used in the bacterial sulfur system 208. In some embodiments, decreasing an amount of buildup may correspondingly decrease an amount of plugging and/or clogging the filter and other materials used in the bacterial sulfur system 208.

Further, once the sulfur fed suspension has been trickled through the filter material and collected in a reservoir, the sulfur fed suspension may be recirculated a number of times through the filter material (e.g., for a time period of—for instance—several weeks) to have satisfying levels of microorganisms and biologically produced sulfuric acid. In some embodiments, once the process is stable; for example, satisfying conversion of sulfur and production of biologically produced sulfuric acid take place, biologically produced sulfuric acid may be withdrawn as a product. In some embodiments, the biologically produced sulfuric acid may reach a predetermined threshold in the sulfur fed suspension prior to the biologically produced sulfuric acid being withdrawn. For example, the predetermined threshold of the biologically produced sulfuric acid may include one or more ranges that may include a certain weight percentage of the sulfur fed suspension—e.g., 0.1 weight (wt.) %, 0.5 weight (wt.) %, 1.0 weight (wt.) %, 1.5 weight (wt.) %, 2.0 weight (wt.) %, 2.5 weight (wt.) %, 3.0 weight (wt.) %, and 3.5 weight (wt.) % of the sulfur fed suspension.

In some embodiments, the filter material may be made of a suitable plastic that may allow adherence of microorganisms. For example, suitable plastics for the filter may include polyethylene or polypropylene or a combination thereof. Continuing the example, polyethylene or polypropylene may be used because they provide sufficient strength and flexibility and because microorganisms (e.g., microorganisms 210) may adhere well to polyethylene and/or polypropylene. Further, because of these advantages, relatively thin sheets of material can be used, so that a higher surface per unit of volume can be used to be covered with microorganisms. Notwithstanding the example of polyethylene and polypropylene, the filter material may include any filter material suitable for bio trickling processes. Other filter materials are also contemplated herein such as, for example, the filters and methods described in and/or with respect to U.S. Pat. No. 10,981,112 titled “FILTER MATERIAL, DEVICE AND METHOD FOR PURIFYING GASES AND LIQUIDS,” which is incorporated in the present disclosure by reference in its entirety.

In some embodiments, the filter structure may include a first sheet of a plastic and a second sheet of plastic to which microorganisms may attach. In these and other embodiments, the first sheet of a plastic may be a flat surface and the second sheet of plastic may be configured in a set of waves in parallel. In these and other embodiments, the first sheet of plastic and the second sheet of plastic may be attached. For example, the surface of the first sheet of plastic may be attached at one or more of the peaks of the waves such that one or more channels may be created between the surfaces of the first sheet of plastic and the second sheet of plastic. In these and other embodiments, the filter may be rolled-up as a cylinder with open pores that are formed by the one or more channels that extend along the longitudinal axis of the filter.

In some embodiments, the reservoir into which the trickled sulfur fed suspension is collected may be positioned downstream of the filter material. In some embodiments, the reservoir may be contained in a unit that is the same for both the reservoir and the filter material so that gravity may assist in the trickle down of the sulfur fed suspension through the filter material to the reservoir. In these and other embodiments, the reservoir, into which the sulfur fed suspension may be collected, may be aerated. In some embodiments, the aerated reservoir may allow for an increase in a saturation amount of sulfur in the sulfur fed suspension, in part, because the sulfur 102 may remain in the suspension throughout the one or more operations performed by the bacterial sulfur system 108. As used throughout the present disclosure, the term “aeration” may include one or more procedures by which oxygen may be added to the sulfur fed suspension. For example, aeration may involve injecting air to the sulfur fed suspension—e.g., injecting air using injectors and/or spargers.

In some embodiments, the environment 200 as described herein may be an example embodiment of one or more systems that may organically generate biologically produced sulfuric acid. Other systems are also contemplated herein such as, for example, the system(s) described in and/or with respect to U.S. Publication No. 2020/0199629 titled “METHOD FOR REMOVAL OF HARMFUL SULPHUROUS COMPOUNDS FROM GAS MIXTURES,” U.S. Pat. No. 10,493,402 titled “METHOD AND APPARATUS FOR REMOVAL OF HYDROGEN SULPHIDE FROM GAS MIXTURES WITH MICROORGANISMS,” U.S. Pat. No. 6,283,309 titled “DEVICE FOR PURIFYING GASES, SUCH AS AIR IN PARTICULAR, OR LIQUIDS, SUCH AS WATER IN PARTICULAR,” U.S. Pat. No. 6,194,198 titled “DEVICE FOR PURIFYING GASES, SUCH AS AIR IN PARTICULAR, OR LIQUIDS, SUCH AS WATER IN PARTICULAR, AND METHOD FOR USE WITH THE DEVICE,” U.S. Pat. No. 5,445,660 titled “APPARATUS FOR CLEANING GASES WITH THE AID OF ORGANIC FILTRATION MATERIAL,” U.S. Publication No. 2010/0089818 titled “DEVICE FOR PURIFYING GASES, SUCH AS AIR IN PARTICULAR, OR LIQUIDS, SUCH AS WATER IN PARTICULAR” each of which is incorporated in the present disclosure by reference in its entirety.

FIG. 3 illustrates an example environment 300 to generate a filtrate 308 using one or more organic feedstocks 302, in accordance with some embodiments of the present disclosure. In the illustrated example, the environment 300 may include a coarse solids removal system 304 and/or a fine solids separation system 306. In some embodiments, the coarse solids separation system 304 and/or the fine solids separation system 306 may include one or more systems and/or processes configured to perform one or more operations on the organic feedstock 302 to generate the filtrate 308.

In some embodiments, the organic feedstock 302 may include one or more organic wastes and/or organic digestates. In some embodiments, the organic feedstock 302 may include animal manure (e.g., cow manure, chicken manure, turkey manure, swine manure, and/or other animal manure), animal waste (e.g., fish emulsion, bones, blood, tissue, and other animal waste), green waste (e.g., kelp, long beans, algae, duckweed, grasses, soy derivatives, and other green waste), and/or any other organic material that may be derived from animals and/or plants. In some embodiments, the organic feedstock 302 may include one or more forms of waste that may include high concentrations of one or more compounds (e.g., ammoniacal nitrogen and/or other plant nutrients) useful, in some circumstances, in extracting, concentrating, and using as a form of liquid or solid fertilizer. In some embodiments, reference to organic feedstock (e.g., the organic feedstock 302) may refer to solutions, solids, compounds, etc. that may include one or more materials and/or components that may have been derived from the organic feedstock. In some embodiments, the organic feedstock may undergo one or more pretreatment steps to prepare the organic feedstock 302 for use to extract, concentrate, and/or use the high concentrations of one or more compounds as a liquid or solid fertilizer.

The coarse solids separation system 304 and/or the fine solids separation system 306 may be configured to perform one or more operations on the organic feedstock 302 to prepare the organic feedstock 302 in such a way that one or more compounds useful as a fertilizer may be extracted and/or used. In these and other embodiments, the coarse solids separation system 304 and/or the fine solids separation system 306 may include one or more devices, systems, and/or processes that may be configured to perform one or more operations on an organic feedstock 302 to generate a filtrate 308.

The coarse solids separation system 304 may be configured to separate and/or remove coarse or large solids from the organic feedstock 302. In these and other embodiments, separating large and/or coarse solids from the organic feedstock may be performed by any instrument capable of separating solids. By way of example and not limitation, the organic feedstock 302 may be introduced to a slope screen, a screw press, a centrifuge, a rotary screen, and/or other mechanical process of separating large solids found in the organic feedstock 302. In these and other embodiments, the separated coarse solids may be composted, sent on to further treatment, or recycled back to an anaerobic digester system, for example.

Additionally or alternatively, the fine solids separation system 306 may include one or more systems and/or processes designed to remove solids finer than the coarse solids removed by the coarse solids separation system 304 from the organic feedstock 302. In these and other embodiments, separating small and/or fine solids from the organic feedstock may be performed by any instrument capable of separating solids. By way of example and not limitation, finer solids may be removed using a membrane, screen, and/or other filter designed to remove fine solids from the organic feedstock 302 that may remain after removal of the coarse solids. In these and other embodiments, the separated fine solids may be composted, be sent on to further treatment, or be recycled back to another pretreatment facility, system, process, device, etc. that may be configured to further degrade solids in the organic feedstock 302 (e.g., an anaerobic digester system). Systems and/or processes have been contemplated herein with respect to using an anaerobic digester to separate solids from wastewater such as, for example, U.S. Publication No. 2015/0008181 titled “WASTEWATER TREATMENT USING CONTROLLED SOLIDS INPUT TO AN ANAEROBIC DIGESTER” which is incorporated herein by reference in its entirety.

In some embodiments, other pretreatment processes may be performed on the organic feedstock 302 other than those performed using the coarse solids separation system 304 and/or the fine solids separation system 306. In some embodiments, the other pretreatment processes may use a biological and/or a thermal hydrolysis process that may further degrade one or more organic solids in the organic feedstock 302. In some embodiments, the use of biological and/or thermal hydrolysis processes may increase one or more of a biogas production and a higher ammoniacal nitrogen recovery. Example methods and systems of pretreating a digestate are additionally contemplated herein such as, for example, systems and processes explained in and/or with respect to U.S. Publication No. US 2020/0277637 titled “CONVERSION OF LIGNOCELLULOSIC BIOMASS INTO BIOGAS,” U.S. Pat. No. 10,800,667 titled “SYSTEM AND METHOD FOR MULTI-FUNCTIONAL SLURRY PROCESSING,” U.S. Publication No. 2021/0094842 titled “SYSTEM AND METHOD FOR MULTI-FUNCTIONAL SLURRY PROCESSING,” and U.S. Pat. No. 9,994,471 titled “LIVESTOCK WASTEWATER TREATMENT SYSTEM AND METHOD” each of which is incorporated in the present disclosure by reference in its entirety. In these and other embodiments, one or more of the coarse solids separation system 304, the fine solids separation system 306, and/or another pretreatment process, device, or system may additionally add fluid to the organic feedstock 302 to produce the filtrate 308.

The filtrate 308 as described in this disclosure may include one or more components of the organic feedstock 302. In some embodiments, the one or more components of the organic feedstock 302 may include materials, compounds, or other components that may be derived, in whole or in part, from the organic feedstock 302. In some embodiments, the filtrate 308 may include a solution including one or more components of the organic feedstock 302 and water such that the filtrate may be passed over a forward osmosis membrane. In some embodiments, while coarse and/or fine solids may have been removed from the organic feedstock 302 to produce the filtrate 308, solids may still be present in the filtrate 308. For example, the filtrate 308 may include solids suspended in a solution including water and the organic feedstock 302. Further continuing the example, solids may have been dissolved in the water that may remain with the filtrate 308. In these and other embodiments, remaining solids in the filtrate 308 may be selectively separated from the water and other compounds using a forward osmosis system (e.g., the forward osmosis system 404).

In these and other embodiments, the filtrate 308 may be used in the forward osmosis system 404 and/or reverse osmosis system 410 to generate one or more fertilizer products as described and illustrated further with respect to FIG. 4 in the present disclosure. In other embodiments, the filtrate may be used in any fertilizer generating system or in any fertilizer generating process in order to obtain a fertilizer from the filtrate.

In some embodiments, an acid may be introduced to the filtrate 308 to adjust the pH of the filtrate 308. In some embodiments, adjusting the pH of the filtrate 308 may increase the stability of the filtrate 308. In some embodiments, adjusting the pH using an acid may aid in recovering or concentrating nitrogen and other plant nutrients. For example, biologically produced sulfuric acid may be added to the filtrate 308. Continuing the example, adding the biologically produced sulfuric acid may increase an eventual amount of recovery of nitrogen from the filtrate 308 (e.g., recovery via forward osmosis described in greater detail with respect to the forward osmosis system 404 described and illustrated with respect to FIG. 4 in the present disclosure). Further, in another example, a bacterial solution produced in an apparatus including oxidized sulfur (e.g., a solution including the organic, biologically produced sulfuric acid 212 described and illustrated further in FIG. 2 in the present disclosure) may be added to the organic feedstock 302 and/or the filtrate 308 to adjust its pH.

FIG. 4 illustrates an example environment 400 of generating an organic fertilizer product 414 and/or an enhanced organic fertilizer product 416 using a filtrate 402 and a draw solution 406, in accordance with some embodiments of the present disclosure. In the illustrated example, the environment 400 may include a forward osmosis system 404 and a reverse osmosis system 410. In some embodiments, the forward osmosis system 404 may perform one or more operations to separate water and other compounds from the filtrate 402 using the draw solution 406. The separation of water and other compounds from the filtrate 402 may produce a forward osmosis concentrate 408. Further, the reverse osmosis system 410, may perform one or more operations to separate out a reverse osmosis permeate 412 from the draw solution 406 to produce an organic fertilizer product 414. In some embodiments, at least a portion of the organic fertilizer product 414 may be recycled as recycled concentrate 418 to re-concentrate the draw solution 406. Additionally or alternatively, the illustrated example environment 400 may include adding and/or combining the forward osmosis concentrate 408 and the organic fertilizer product 414 which may create an enhanced organic fertilizer product 416.

The filtrate 402 may be the same as and/or include the filtrate 308 described and illustrated with respect to FIG. 3 in the present disclosure. In some embodiments, the filtrate 402 may include a solution including one or more components of an organic waste (e.g., organic feedstock 302 described with respect to FIG. 3 in the present disclosure) and water such that the filtrate may be passed over a forward osmosis membrane as described further with respect to the forward osmosis system 404. In these and other embodiments, remaining solids in the filtrate 402 may be selectively separated from the water and other compounds using a forward osmosis system (e.g., the forward osmosis system 404).

The forward osmosis system 404 may include one or more systems, processes, devices, etc. that may be configured to perform one or more operations on the filtrate 402 to further concentrate any remaining solids (e.g., solid, or suspended solids) in the filtrate 402 by separating the water, ammonia, and other compounds from the filtrate 402. In some embodiments, separating the water, ammonia, and other compounds from the filtrate 402 may be performed using a forward osmosis membrane and the draw solution 406, where the draw solution 406 may create osmotic potential across the forward osmosis membrane through which water, ammonia, and other compounds may travel from the filtrate 402 to the draw solution 406. For example, using the draw solution 406 may provide more than 90% water removal with no further energy input to the process than that required to bring both the filtrate 402 to one side of a forward osmosis membrane and the draw solution 406 to the other side of the forward osmosis membrane.

In some embodiments, the forward osmosis system 404 may include one or more processes that may use an osmotic membrane to further concentrate the filtrate 402. In these and other embodiments, because the driving force causing the transfer of mass (e.g., water, ammonia, and/or other compounds) through the forward osmosis membrane is osmotic pressure, no additional energy input may be required to cause the transfer to occur beyond what is required to place the filtrate 402 and the draw solution 406 in contact with the forward osmosis membrane (e.g., through one or more pumps, etc.). In these and other embodiments, the forward osmosis system 404 may operate at low pressures (typically 1 to 2 Bar), thereby reducing fouling of the forward osmosis membrane. In some embodiments, water may move from the filtrate 402 to the draw solution 406 due to a concentration gradient and not due to applied pressure or heat or any other power input. In some embodiments, water will diffuse through the membrane from the filtrate 402 to the draw solution 406. In some embodiments, material, structure, and/or arrangement of the forward osmosis membrane may affect an amount and/or rate of transfer of water and other compounds through the forward osmosis membrane.

In some embodiments, the forward osmosis membrane may include one or more membranes that may allow for separation of water and other compounds from the filtrate due to osmotic pressure. In some embodiments, the forward osmosis membrane may be a semipermeable thin film composite membrane. For example, the forward osmosis membrane may include one or more of a fiber, plate and frame, and spiral wound membrane configuration, a semipermeable cellulosic membrane in one of a fiber, plate and frame, and spiral wound membrane configuration, or one or more combinations of the aforementioned forward osmosis membrane configurations. In some embodiments, the forward osmosis membrane may include one or membranes such as, for example, the one or more forward osmosis membranes described in and/or with respect to U.S. Pat. No. 10,835,870 titled “METHODS OF MANUFACTURING A MULTI-LEAF MEMBRANE MODULE AND MULTI-LEAF MEMBRANE MODULES,” and U.S. Publication No. 2013/0186824 titled “SPIRAL CROSS FLOW MEMBRANE FILTRATION DEVICE AND PROCESS” each of which is incorporated in the present disclosure by reference in its entirety.

In some embodiments, the draw solution 406 may be used to create osmotic pressure through the forward osmosis membrane to filter the water, ammonia, and other compounds from the filtrate 402. For example, the filtrate 402 may be used in a first stream on a first side of the forward osmosis membrane and the draw solution 406 may be used as a second stream on a second side of the forward osmosis membrane. Continuing the example, the draw solution 406 may create an amount of osmotic pressure sufficient to separate water, ammonia, and other compounds from the filtrate 402.

In some embodiments, the draw solution 406 may include an aqueous solution that may include biologically produced sulfuric acid. For example, the draw solution 406 may include the organic, biologically produced sulfuric acid 212 as described and illustrated with respect to FIG. 2 in the present disclosure. In these and other embodiments, the use of the organic, biologically produced sulfuric acid 212 in the draw solution 406 may separate one or more of ammonia, water, and other compounds from the filtrate 402. In some embodiments, the ammonia may form ammonium salt in the draw solution 406. In some embodiments, the separation of ammonia, water, and other compounds from the filtrate 402 may correspondingly increase a concentration of nitrogen, potassium, phosphorus, salts, and/or other solids, etc. remaining in the filtrate 402. In some embodiments, matter remaining—including the increased concentration of solids, nitrogen, potassium, phosphorus, salts, etc.—in the filtrate 402 may be included in the forward osmosis concentrate 408.

In some embodiments, ammonia, water, and other compounds may be separated through the forward osmosis membrane to the draw solution 406, leaving one or more solids and/or compounds behind in the filtrate 402. In these and other embodiments, the remaining solids and/or other compounds from the filtrate 402 may be referred to herein as the forward osmosis concentrate 408. In some embodiments, the forward osmosis concentrate 408 may include nitrogen, phosphorus, potassium, and/or other trace minerals. In some embodiments, the forward osmosis concentrate 408 may be recycled back to a system that may treat one or more organic wastes; for example, the pretreatment system 108 described in FIG. 1, the coarse solids separation system 304, and/or the fine solids separation system 306 as described in FIG. 3 in the present disclosure. Additionally or alternatively, the forward osmosis concentrate 408 may be used for irrigation, compost, or as a fertilizer containing nitrogen, phosphorus, potassium, and other trace minerals. In some embodiments, the forward osmosis concentrate 408 may be added to an organic fertilizer product (e.g., the organic fertilizer product 414) to produce a fertilizer product with added micronutrients (e.g., enhanced fertilizer product 416). In these and other embodiments, the forward osmosis concentrate 408 and/or the one or more processes, devices, and/or products used to generate the forward osmosis concentrate 408 may be certified as organic.

In some embodiments, because water and other compounds may be separated through the forward osmosis membrane from the filtrate 402 to the draw solution 406, the draw solution 406 may become diluted. Further, because the draw solution 406 may become progressively diluted, the osmotic pressure across the forward osmosis membrane may correspondingly decrease. In some embodiments, to continue to maintain the draw solution 406 at a concentration sufficient to extract water and other compounds from the filtrate 402 through the forward osmosis membrane, more draw solution 406 may be added for use in the forward osmosis system 404. In some embodiments, the draw solution 406 that may be added may include the organic, biologically produced sulfuric acid 212 as described and illustrated with respect to FIG. 2 in the present disclosure. Additionally or alternatively, water may be extracted from the draw solution 406 via a reverse osmosis system—e.g., reverse osmosis system 410. In some embodiments, by extracting water from the draw solution 406, the draw solution 406 may correspondingly increase in concentration and may offset water extracted from the filtrate 402 and added to the draw solution 406.

Additionally or alternatively, other forward osmosis systems and/or methods of producing purified water and other liquids through forward osmosis are also contemplated herein such as, for example, the system(s) described in and/or with respect to U.S. Pat. No. 11,040,904 titled “METHODS AND SYSTEMS FOR TREATING WASTEWATER VIA FORWARD OSMOSIS,” U.S. Publication No. 2012/0231535 titled “ORGANIC FORWARD OSMOSIS SYSTEM,” U.S. Publication No. 2020/0196620 titled “OSMOTIC MILK CONCENTRATOR HAVING A NUTRIENT FORTIFIED DRAW SOLUTION,” U.S. Publication No. 2022/0047991 titled “STORAGE PROTECTION FOR FORWARD OSMOSIS HYDRATION OR DEWATERING SYSTEM,” U.S. Publication No. 2015/0360983 titled “WATER REUSE SYSTEM AND METHOD,” U.S. Publication No. 2015/0014248 titled “METHOD AND SYSTEM FOR GENERATING STRONG BRINES,” U.S. Pat. No. 7,303,674 titled “FORWARD OSMOSIS PRESSURIZED DEVICE AND PROCESS FOR GENERATING POTABLE WATER,” and U.S. Pat. No. 6,849,184 titled “FORWARD OSMOSIS PRESSURIZED DEVICE AND PROCESS FOR GENERATING POTABLE WATER,” each of which is incorporated in the present disclosure by reference in its entirety.

The reverse osmosis system 410 includes one or more systems, devices, and/or processes that may be configured to perform one or more operations using the draw solution 406. In some embodiments, the reverse osmosis system 410 may be used in conjunction with the forward osmosis system 404. For example, the forward osmosis system 404 may extract water and other compounds from a filtrate 402 using a draw solution 406. Continuing the example, the draw solution 406 used to extract the water and other compounds becomes diluted over time as water is added to the draw solution 406. Further, the diluted draw solution 406 may then be cycled through the reverse osmosis system 410 to extract some of the added water and increase the concentration of the draw solution 406 used in the forward osmosis system 404.

In some embodiments, the draw solution 406—that may be diluted—may be pumped and/or otherwise fed under pressure to the reverse osmosis system 410 where the draw solution 406 may be re-concentrated to appropriate draw strength, and the clean product water is simultaneously produced (e.g., the reverse osmosis permeate 412). In some embodiments, the reverse osmosis system 410 may produce the reverse osmosis permeate 412 that may include a purified water stream that may be reused.

In some embodiments, the reverse osmosis system 410 may include one or more reverse osmosis membranes and/or membranes that may be configured to separate the reverse osmosis permeate 412 from the draw solution 406. In these and other embodiments, after extracting the reverse osmosis permeate 412 from the draw solution 406, a solution that may include ammonia, ammonium salt, and other compounds remains. In some embodiments, the remaining ammonia, ammonium salt, and other compounds may be the recycled concentrate 418. In these and other embodiments, the recycled concentrate 418 including ammonia and/or ammonium salt may be recycled and used in the draw solution 406 to maintain sufficient draw strength in the forward osmosis system 404. In some embodiments, the recycled concentrate 418 may be siphoned off and used as a fertilizer product (e.g., organic fertilizer product 414). In these and other embodiments, the organic fertilizer product 414 may be certifiably organic. In some embodiments, a first portion of the remaining solution may be recycled back to re-concentrate the draw solution 406 used in the forward osmosis system 404. Further, a second portion of the remaining solution may be extracted as the organic fertilizer product 414.

Other systems and/or processes that may be used to extract ammonia and/or ammonium salt from an aqueous solution have been contemplated as part of the present disclosure such as, for example, systems and processes described in and with respect to U.S. Pat. No. 10,793,458 titled “PROCESS TO RECOVER AMMONIUM BICARBONATE FROM WASTEWATER,” U.S. Pat. No. 10,106,447 titled “PROCESS TO RECOVER AMMONIUM BICARBONATE FROM WASTEWATER,” U.S. Pat. No. 10,604,432 titled “PROCESS TO RECOVER AMMONIUM BICARBONATE FROM WASTEWATER,” U.S. Pat. No. 11,254,581 titled “PROCESS TO RECOVER AMMONIUM BICARBONATE FROM WASTEWATER,” U.S. Publication No. 2022/0227637 titled “PROCESS TO RECOVER AMMONIUM BICARBONATE FROM WASTEWATER,” and U.S. Publication No. 2014/0091040 titled “BICARBONATE CONVERSION ASSISTED RO TREATMENT SYSTEM FOR NATURAL GAS FLOWBACK WATER,” each of which is incorporated in the present disclosure by reference in its entirety.

This integration of forward osmosis processes and reverse osmosis processes into a single system and/or method may achieve a level of nutrient (nitrogen to phosphorus) balancing of fertilizer production from agricultural biomass digesters (e.g., the organic feedstock 302) while also harvesting potentially potable water grade water from the process.

In some embodiments, the organic fertilizer product 414 may be combined with the forward osmosis concentrate 408 to generate the enhanced organic fertilizer product 416. For example, the organic fertilizer product 414 may include ammonia and/or ammonium salt which may be used as a fertilizer. Continuing the example, the forward osmosis concentrate 408 from the forward osmosis system 404 may be added to the organic fertilizer product 414, thereby adding one or more of potassium, phosphorus, nitrogen, and/or other trace minerals. In these and other embodiments, the addition of the forward osmosis concentrate 408 to the organic fertilizer product 414 may produce the enhanced organic fertilizer product 416. In these and other embodiments, the enhanced organic fertilizer product 416 may be certified as organic. In some embodiments, the organic fertilizer product 414, the enhanced organic fertilizer product 416, and/or the forward osmosis concentrate 408, that may be used as a fertilizer product, may include, for example, systems, products and/or processes described in and/or with respect to U.S. Pat. No. 10,023,501 titled “ORGANIC LIQUID FERTILIZER AND PROCESS OF MAKING,” and U.S. Pat. No. US 10,889,528 titled “ORGANIC LIQUID FERTILIZER,” both of which are incorporated in the present disclosure by reference in their entirety. Further, in some embodiments, other methods of recovering ammonia from the draw solution are contemplated herein such as, for example, the methods and/or processes described in and/or with respect to U.S. Publication No. 2021/0162343 titled “METHOD AND DEVICE FOR REMOVING AMMONIA FROM EXHAUST AIR FROM A LIVESTOCK STABLE,” U.S. Pat. No. 10,889,528 titled “ORGANIC LIQUID FERTILIZER,” U.S. Pat. No. 10,556,837 titled “ORGANIC LIQUID FERTILIZER,” and U.S. Pat. No. 10,023,501 titled “ORGANIC LIQUID FERTILIZER AND PROCESS OF MAKING,” each of which is incorporated in the present disclosure by reference in its entirety.

In some embodiments, the environment 400 as described herein may be an example embodiment of one or more systems that may generate an organic fertilizer product. Other systems are also contemplated herein such as, for example, the system(s) described in and/or with respect to U.S. Publication No. 2012/0231535 titled “ORGANIC FORWARD OSMOSIS SYSTEM” which is incorporated in the present disclosure by reference in its entirety.

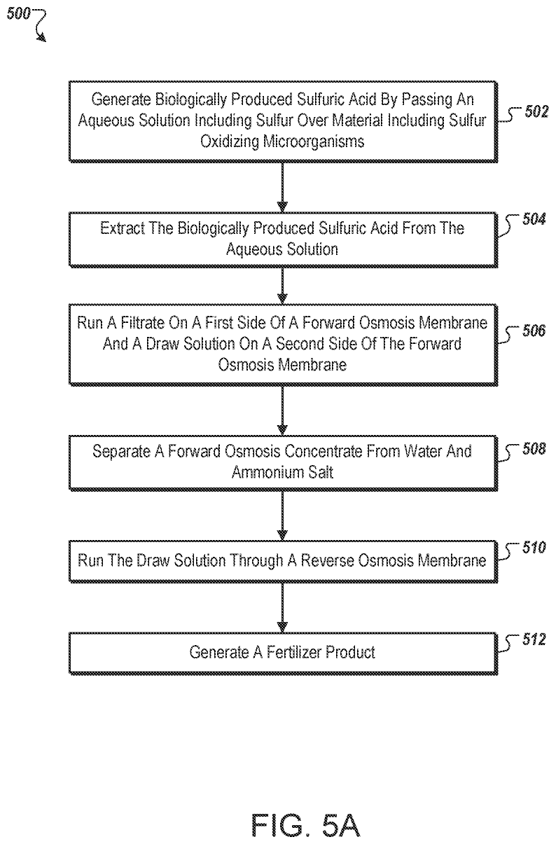

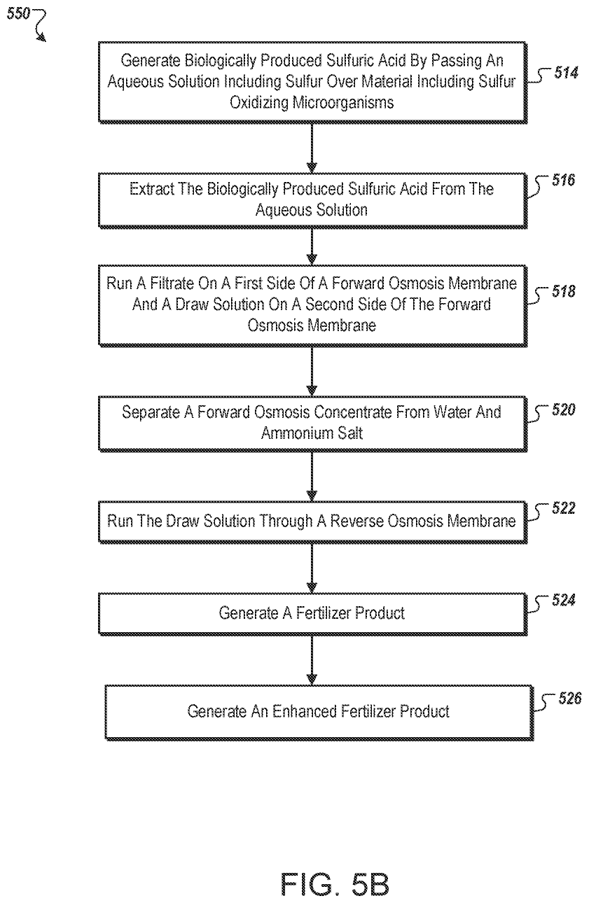

FIG. 5A illustrates a flowchart of an example method 500 of generating fertilizer material, in accordance with at least one embodiment described in the present disclosure. The method 500 may be performed by any suitable system, apparatus, or device as described in the present disclosure. The method 500 may include one or more blocks 502, 504, 506, 508, 510, and 512. Although illustrated with discrete blocks, the steps and operations associated with one or more of the blocks of the method 500 may be divided into additional blocks, combined into fewer blocks, or eliminated, depending on the particular implementation.

In some embodiments, the method 500 may start at block 502. At the block 502, biologically produced sulfuric acid may be generated by passing an aqueous solution that may include sulfur over material that may include sulfur oxidizing microorganisms. In some embodiments, the material that the aqueous solution may be passed over may include one or more filters. In some embodiments, the material may include a biofilm of microorganisms where the microorganisms may oxidize the sulfur that may be included in the aqueous solution. In some embodiments, the microorganisms may produce, grow, and/or otherwise generate biologically produced sulfuric acid using the sulfur in the aqueous solution. Further, in some embodiments, the sulfur included in the aqueous solution may be pretreated. In these and other embodiments, pretreating the sulfur particles may include decreasing the particle size of at least a portion of the sulfur particles to include a lateral dimension of fifteen (15) microns or less as described, for example with respect to the pretreatment system 108 in FIG. 1, the coarse solids separation system 304, and/or the fine solids separation system 306 described and illustrated with respect to FIG. 3. In some embodiments, the aqueous solution including the sulfur may be recirculated over the material (e.g., the one or more filters) that may include the biofilm of microorganisms. In some embodiments, the aqueous solution may be recirculated over the material until the biologically produced sulfuric acid in the aqueous solution reaches a predefined threshold (e.g., at least 0.1% weight, 0.5% weight, 1.0% weight, 1.5% weight, 2.0% weight, 2.5% weight, 3.0% weight, and/or 3.5% weight). In these and other embodiments, the aqueous solution may additionally include microorganisms and the aqueous solution may be passed through a filter and, eventually, a biofilm may form on the filter over time. These and other embodiments may be described and illustrated further with respect to the bacterial sulfur system 208 described in FIG. 2 in the present disclosure.

At block 504, biologically produced sulfuric acid may be extracted from the aqueous solution. In some embodiments, biologically produced sulfuric acid may be extracted from the aqueous solution once the amount of biologically produced sulfuric acid in the aqueous solution has reached a predefined threshold. In some embodiments, once the amount of biologically produced sulfuric acid has reached the predefined threshold, part of the aqueous solution may be siphoned off. In these and other embodiments, the aqueous solution may continually be siphoned off and more aqueous solution including sulfur may be continually added to the aqueous solution, described further in the environment 200 and/or the bacterial sulfur system 208 described in FIG. 2 in the present disclosure.

At block 506, a filtrate may be run on a first side of a forward osmosis membrane and a draw solution may be run on a second side of the forward osmosis membrane. In these and other embodiments, the forward osmosis membrane may include a semi-permeable membrane where water and other compounds may transfer through the membrane due to osmotic pressure that may be created by one or more differences between the filtrate on the first side of the membrane and the draw solution on the second side of the membrane. In some embodiments, the filtrate may include an organic feedstock (e.g., food waste, green waste, etc.). In some embodiments, the draw solution may include the biologically produced sulfuric acid that may have been generated using sulfur oxidizing microorganisms. These and other embodiments may be described further with respect to the forward osmosis system 404 described in FIG. 4 in the present disclosure.

At block 508, a forward osmosis concentrate may be separated from at least water and ammonium salt. In some embodiments, water, ammonia, and other compounds from the filtrate may pass through the membrane to the draw solution. In these and other embodiments, at least water and ammonia may pass from the filtrate to the draw solution. In some embodiments, the forward osmosis concentrate may include one or more of phosphorus, potassium, nitrogen, and other compounds that may be used as fertilizer. In some embodiments, the ammonia may form ammonium salt in the draw solution. The forward osmosis concentrate may be further described with respect to the forward osmosis system 404 and the forward osmosis concentrate 408 described with respect to FIG. 4 in the present disclosure.

At block 510, the draw solution may be run through a reverse osmosis membrane. In some embodiments, the draw solution may become diluted from the water that may pass through from the filtrate to the draw solution through the forward osmosis membrane. In these and other embodiments, the draw solution may be run through one or more reverse osmosis membranes and/or filters to separate water from the draw solution. In these and other embodiments, the draw solution may be reconcentrated to include one of ammonia, ammonium salt, biologically produced sulfuric acid, etc. In some embodiments, ammonia, ammonium salt, biologically produced sulfuric acid, and/or other compounds may be re-added to the draw solution in the forward osmosis process to reconcentrate the draw solution. In some embodiments, re-concentrating the draw solution may increase the draw strength and/or osmotic potential between the draw solution and the filtrate that may drive the mass transfer of water and other compounds from the filtrate to the draw solution. In some embodiments, the remaining compounds in the draw solution after being run through one or more reverse osmosis membranes may include one or more compounds and/or materials that may be used as a fertilizer product. These and other embodiments may be described in further detail with respect to the reverse osmosis system 410 described with respect to FIG. 4 in the present disclosure.

At block 512, a fertilizer product may be generated. In some embodiments, the fertilizer product may include the fertilizer product produced from the draw solution and the forward osmosis concentrate. In these and other embodiments, the forward osmosis concentrate, and the fertilizer product may be used independently as fertilizer materials. These and other embodiments may be described in further detail with respect to the organic fertilizer product 414 and/or the forward osmosis concentrate 408 described with respect to FIG. 4 in the present disclosure.

Modifications, additions, or omissions may be made to the method 500 without departing from the scope of the present disclosure. For example, the operations of method 500 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

FIG. 5B illustrates a flowchart of an example method 550 of generating one or more of a fertilizer product and an enhanced fertilizer product, in accordance with at least one embodiment described in the present disclosure. The method 550 may be performed by any suitable system, apparatus, or device as described in the present disclosure. The method 550 may be an extension of the method 500 described with respect to FIG. 5A. The method 550 may include one or more blocks 514, 516, 518, 520, 522, 524, and 526. Although illustrated with discrete blocks, the steps and operations associated with one or more of the blocks of the method 550 may be divided into additional blocks, combined into fewer blocks, or eliminated, depending on the particular implementation.

In some embodiments, the method 550 may start at block 514. At the block 514, biologically produced sulfuric acid may be generated by passing an aqueous solution that may include sulfur over material that may include sulfur oxidizing microorganisms. In some embodiments, the material that the aqueous solution may be passed over may include one or more filters. In some embodiments, the material may include a biofilm of microorganisms where the microorganisms may oxidize the sulfur that may be included in the aqueous solution. In some embodiments, the microorganisms may produce, grow, and/or otherwise generate biologically produced sulfuric acid using the sulfur in the aqueous solution. Further, in some embodiments, the sulfur included in the aqueous solution may be pretreated. In these and other embodiments, pretreating the sulfur particles may include decreasing the particle size of at least a portion of the sulfur particles to include a lateral dimension of fifteen (15) microns or less as described, for example with respect to the pretreatment system 108 in FIG. 1, the coarse solids separation system 304, and/or the fine solids separation system 306 described and illustrated with respect to FIG. 3. In some embodiments, the aqueous solution including the sulfur may be recirculated over the material (e.g., the one or more filters) that may include the biofilm of microorganisms. In some embodiments, the aqueous solution may be recirculated over the material until the biologically produced sulfuric acid in the aqueous solution reaches a predefined threshold (e.g., 0.1% weight, 0.5% weight, 1.0% weight, 1.5% weight, 2.0% weight, 2.5% weight, 3.0% weight, and/or 3.5% weight). In these and other embodiments, the aqueous solution may additionally include microorganisms and the aqueous solution may be passed through a filter and, eventually, a biofilm may form on the filter over time. These and other embodiments may be described and illustrated further with respect to the bacterial sulfur system 208 described in FIG. 2 and at block 502 described with respect to FIG. 5A in the present disclosure.

At block 516, biologically produced sulfuric acid may be extracted from the aqueous solution. In some embodiments, biologically produced sulfuric acid may be extracted from the aqueous solution once the amount of biologically produced sulfuric acid in the aqueous solution has reached a predefined threshold. In some embodiments, once the amount of biologically produced sulfuric acid has reached the predefined threshold, part of the aqueous solution may be siphoned off In these and other embodiments, the aqueous solution may continually be siphoned off and more aqueous solution including sulfur may be continually added to the aqueous solution, described further in the environment 200 and/or the bacterial sulfur system 208 described in FIG. 2 and at block 504 described with respect to FIG. 5A in the present disclosure.

At block 518, a filtrate may be run on a first side of a forward osmosis membrane and a draw solution may be run on a second side of the forward osmosis membrane. In these and other embodiments, the forward osmosis membrane may include a semi-permeable membrane where water and other compounds may transfer through the membrane due to osmotic pressure that may be created by one or more differences between the filtrate on the first side of the membrane and the draw solution on the second side of the membrane. In some embodiments, the filtrate may include an organic feedstock (e.g., food waste, green waste, etc.). In some embodiments, the draw solution may include the biologically produced sulfuric acid that may have been generated using sulfur oxidizing microorganisms. These and other embodiments may be described further with respect to the forward osmosis system 404 described in FIG. 4 and at block 506 described with respect to FIG. 5A in the present disclosure.

At block 520, a forward osmosis concentrate may be separated from at least water and ammonium salt. In some embodiments, water and other compounds from the filtrate may pass through the membrane to the draw solution. In these and other embodiments, at least water and ammonia may pass from the filtrate to the draw solution. In some embodiments, the forward osmosis concentrate may include one or more of phosphorus, potassium, nitrogen, and other compounds that may be used as fertilizer. The forward osmosis concentrate may be further described with respect to the forward osmosis system 404 and the forward osmosis concentrate 408 described with respect to FIG. 4 and at block 508 described with respect to FIG. 5A in the present disclosure.

At block 522, the draw solution may be run through a reverse osmosis membrane. In some embodiments, the draw solution may become diluted from the water that may pass through from the filtrate to the draw solution through the forward osmosis membrane. In these and other embodiments, the draw solution may be run through one or more reverse osmosis membranes and/or filters to separate water from the draw solution. In these and other embodiments, the draw solution may be reconcentrated to include one of ammonia, ammonium salt, biologically produced sulfuric acid, etc. In some embodiments, ammonia, ammonium salt, biologically produced sulfuric acid, and/or other trace compounds may be re-added to the draw solution in the forward osmosis process to reconcentrate the draw solution. In some embodiments, re-concentrating the draw solution may increase the draw strength and/or osmotic potential between the draw solution and the filtrate that may drive the mass transfer of water and other compounds from the filtrate to the draw solution.

At block 524, a fertilizer product may be generated. In some embodiments, the remaining compounds in the draw solution after being run through one or more reverse osmosis membranes may include one or more compounds and/or materials that may be used as a fertilizer product. These and other embodiments may be described in further detail with respect to the reverse osmosis system 410 described with respect to FIG. 4 and at block 512 with respect to FIG. 5A in the present disclosure.

At block 526, an enhanced fertilizer product may be generated. In some embodiments, the enhanced fertilizer product may include at least a portion of the fertilizer product produced from the draw solution (e.g., at block 524 described with respect to FIG. 5B and at block 512 described with respect to FIG. 5A) and at least a portion of the forward osmosis concentrate. In these and other embodiments, the forward osmosis concentrate, and the fertilizer product may be used independently as fertilizer materials. In some embodiments, the combination of the fertilizer product and the forward osmosis concentrate may produce the enhanced fertilizer product that may include one or more of ammonia, ammonium salt, potassium, phosphorus, nitrogen, biologically produced sulfuric acid and other trace minerals, etc. These and other embodiments may be described in further detail with respect to the enhanced organic fertilizer product 416 described with respect to FIG. 4 in the present disclosure.

Modifications, additions, or omissions may be made to the method 550 without departing from the scope of the present disclosure. For example, the operations of method 550 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood that ammonium salt, as used in the present disclosure may refer to one or more ammonium salts, which are fertilizer salts may include a salt anion of at least one of: chloride, nitrate, sulfate, bicarbonate, cyanamide, phosphate, and other fertilizer salt anions. Other fertilizers may include ammoniacal fertilizers, nitrate fertilizers, amide fertilizers, and other fertilizers.

Additionally, the one or more fertilizer salts may include at least one of: potassium chloride, sodium nitrate, calcium nitrate, potassium nitrate, calcium, a bicarbonate, calcium cyanamide, dicalcium phosphate, and other fertilizer salts.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

All references recited herein are incorporated herein by specific reference in their entirety.

What is claimed is:

1. A method of generating organic fertilizer material comprising:
generating biologically produced sulfuric acid by passing an aqueous solution including sulfur over a material including sulfur oxidizing microorganisms;
recirculating the aqueous solution over the material until an amount of biologically produced sulfuric acid in the aqueous solution reaches a predefined threshold;
extracting the biologically produced sulfuric acid from the aqueous solution; and
generating the organic fertilizer material using the extracted biologically produced sulfuric acid and an organic feedstock.

2. The method of claim 1, further comprising:
providing a draw solution including the extracted biologically produced sulfuric acid to a forward osmosis system; and
generating a second organic fertilizer material using the forward osmosis system with the provided draw solution and a filtrate generated using the organic feedstock.

3. The method of claim 1, wherein the predefined threshold is at least 0.1 wt. % of the biologically produced sulfuric acid in the aqueous solution.

4. The method of claim 1, further comprising:
pretreating the sulfur for use in the aqueous solution by decreasing a particle size of at least a portion of sulfur particles in the sulfur.

5. The method of claim 4, wherein decreasing the particle size of the at least a portion of the sulfur particles in the sulfur is performed using one or more of dry milling, wet milling, high shear mixing, and cavitation.

6. The method of claim 1, wherein the sulfur includes one or more of elemental sulfur, sulfur bentonite, hydrogen sulfide, and sulfur dioxide.

7. The method of claim 1, further comprising:
seeding the aqueous solution with the sulfur oxidizing microorganisms;
circulating the aqueous solution with the sulfur oxidizing microorganisms through the material;
aerating the aqueous solution; and
recirculating the aqueous solution through the material to generate a biofilm of sulfur oxidizing microorganisms on the material.

8. The method of claim 2, wherein the forward osmosis system includes:
running the filtrate on a first side of a forward osmosis membrane and running the draw solution including the extracted biologically produced sulfuric acid on a second side of the forward osmosis membrane; and
separating through the forward osmosis membrane, a first stream including a forward osmosis concentrate including one or more of phosphorus, nitrogen, and potassium and a second stream including one or more of water, ammonia, and ammonium salt.

9. The method of claim 8, wherein the forward osmosis concentrate is aerated to precipitate one or more phosphorus compounds.

10. The method of claim 1, wherein processing the organic feedstock further comprises:
separating coarse solids from the organic feedstock; and
separating fine solids from the organic feedstock.

11. The method of claim 10, further comprising:
using one or more organic polymers to further separate at least one of the coarse solids and the fine solids from the organic feedstock.

12. The method of claim 9, further comprising:
pretreating the organic feedstock using at least one of a thermal hydrolysis process and a bacterial process.

13. A method of generating organic fertilizer product comprising:
running a filtrate stream on a first side of a forward osmosis membrane and running a draw solution on a second side of the forward osmosis membrane, the draw solution including biologically produced sulfuric acid generated using sulfur oxidizing microorganisms;
separating through the forward osmosis membrane, a first stream including a forward osmosis concentrate including one or more of phosphorus, nitrogen, and potassium and a second stream including one or more of water and ammonium salt; and
generating a fertilizer product using reverse osmosis including running the second stream through a reverse osmosis membrane producing a third stream and a fourth stream, the third stream including water and the fourth stream including the fertilizer product.

14. The method of claim 13, wherein the fertilizer product includes one or more of ammonia, and ammonium salt.

15. The method of claim 13, further comprising:
reconcentrating the draw solution by adding the second stream including the fertilizer product to the draw solution.

16. The method of claim 13, further comprising:
generating an enhanced fertilizer product by combining at least the forward osmosis concentrate and the fertilizer product.

17. The method of claim 13, wherein the filtrate stream includes at least one of:
an organic feedstock including at least organic waste;
components, materials, or solutions derived from the organic waste; and
solutions separated from the organic waste.

18. The method of claim 17, further comprising:
adding a bacterial solution including oxidized sulfur to adjust a pH of the filtrate stream.

19. The method of claim 13, further comprising at least one of:
aerating the forward osmosis membrane concentrate to precipitate phosphorus compounds;
adding the precipitated phosphorus compounds to the fertilizer product; and
using the precipitated phosphorus compounds as a separate fertilizer product.

20. The method of claim 13, wherein the water in the first stream is reused to create a new filtrate stream including at least the water and an organic feedstock.

21. A method of generating organic fertilizer material comprising:
providing an aqueous solution including sulfur particles, the sulfur particles are pretreated such that at least a portion of the sulfur particles are no greater than fifteen (15) microns;
passing the aqueous solution over a filter including a biofilm of sulfur oxidizing microorganisms, the sulfur oxidizing microorganisms generating biologically produced sulfuric acid from the sulfur in the aqueous solution;
recirculating the aqueous solution over the filter including the biofilm until an amount of biologically produced sulfuric acid in the aqueous solution reaches a predefined threshold;
extracting the biologically produced sulfuric acid from the aqueous solution;
running a filtrate stream on a first side of a forward osmosis membrane, the filtrate stream including an organic feedstock and running a draw solution on a second side of the forward osmosis membrane, the draw solution including the extracted biologically produced sulfuric acid;
separating through the forward osmosis membrane, a first stream including a forward osmosis concentrate including one or more of phosphorus, nitrogen, and potassium and a second stream including at least water and ammonium salt;
running the second stream through one or more reverse osmosis membranes producing water separated out from a fertilizer product; and
generating an enhanced fertilizer product by combining the at least a portion of the forward osmosis concentrate and the fertilizer product.

22. The method of claim 21, wherein the predefined threshold is at least 0.1 wt. % of the biologically produced sulfuric acid in the aqueous solution.

23. The method of claim 21, further comprising:
pretreating the sulfur for use in the aqueous solution by decreasing a particle size of the at least a portion of sulfur particles in the sulfur using one or more of dry milling, wet milling, high shear mixing, and cavitation.

24. The method of claim 21, wherein the sulfur includes one or more of elemental sulfur, sulfur bentonite, hydrogen sulfide, and sulfur dioxide.

25. The method of claim 21, further comprising:
seeding the aqueous solution with the sulfur oxidizing microorganisms;
circulating the aqueous solution with the sulfur oxidizing microorganisms through the filter;
aerating the aqueous solution; and
recirculating the aqueous solution through the filter to generate the biofilm.

26. The method of claim 21, further comprising:
generating the fertilizer product using reverse osmosis including running the second stream through the one or more reverse osmosis membranes producing a third stream and a fourth stream, the third stream including water and the fourth stream including at least ammonium salt.

27. The method of claim 26, wherein the forward osmosis concentrate is aerated to precipitate one or more phosphorus compounds.

28. The method of claim 21, wherein the filtrate stream includes one or more of animal waste, green waste, and food waste.

29. The method of claim 21, wherein processing the organic feedstock further comprises:
separating coarse solids from the organic feedstock using one or more of a screw press, a centrifuge, and a screen; and
separating fine solids from the organic feedstock using one or more of a membrane, a filter, and a screen.

30. The method of claim 21, wherein the organic fertilizer material includes one or more of ammonia, and ammonium salt.
